(19) United States
(12) Patent Application Publication
(10) Pub. No.: US 2010/0247438 A1
(43) Pub. Date: Sep. 30, 2010

(54) IN VITRO METHOD FOR DIAGNOSING TUMOR DISEASES

(76) Inventors: Arne Hengerer, Mohrendorf (DE); Andreas Kappel, Konigstein (DE); Luis Lasalvia, White Plains, NY (US); Andrea Lichte, Marburg (DE); Thomas Mertelmeier, Erlangen (DE); Sven Meyburg, Erlangen (DE); Christoph Petry, Neuss (DE); Marcus Pfister, Bubenreuth (DE); Martin Requardt, Nurnberg (DE)

Correspondence Address: HARNESS, DICKEY & PIERCE, P.L.C., P.O.BOX 8910, RESTON, VA, 20195, US

(21) Appl. No.: 12/731,499

(22) Filed: Mar. 25, 2010

(30) Foreign Application Priority Data
Mar. 31, 2009 (DE) 10 2009 015 784.0

(51) Int. Cl. A61K 49/00; G01N 33/53; G01N 33/573; G01N 33/48; A61B 1/00

(52) U.S. Cl. 424/9.1; 435/7.1; 435/7.4; 436/64; 600/101

(57) ABSTRACT

An in vitro method is for diagnosing a tumor disease in a patient. In at least one embodiment, the method includes: (i) determining an IVD marker or an IVD marker panel in at least one biological sample of a patient, wherein the IVD marker has a high sensitivity to the tumor disease, (ii) determining the proportion of patients tested positive due to an adapted reference range of the IVD marker/IVD marker panel, wherein the reference range was adapted such that the number of individuals with false negative tests, the number of individuals with false positive tests and the number of individuals ultimately needing to be subjected to imaging diagnostics to clarify false negative and false positive results are balanced in respect of one another such that tumor screening can be carried out, possibly: (iii) deciding to carry out an imaging method specific to the respective tumor disease for clarifying possible false negative and/or false positive IVD results, or (iv) repeating stages (i) and (ii) after a defined time interval, or (v) carrying out an imaging method for imaging the tumor.

### PRIORITY STATEMENT

The present application hereby claims priority under 35 U.S.C. §119 on German patent application number DE 10 2009 015 784.0 filed Mar. 31, 2009, the entire contents of which are hereby incorporated herein by reference.

### FIELD

At least one embodiment of the invention generally relates to an in vitro method (in vitro assay or in vitro assay method) for diagnosing tumor diseases/cancer diseases.

### BACKGROUND

In Europe, cancer diseases are the second most common cause of death. Therefore, the early recognition of a tumor or cancer disease is very important so that tumors can be removed before lymph nodes or other organs are affected. At this stage, surgical and systemic treatment approaches have the highest probability of success and can perform more cost-effectively as well than at the later stage.

IVD markers (in vitro diagnostic markers), i.e. tumor markers, are increasingly being used in tumor diagnosis. They also permit efficient progress monitoring and therapy assessment. Further fields of application for determining tumor markers are the examination of groups at risk (e.g. in the case of a positive family history, liver cirrhosis, cryptochidism, gynecological tumors), the differential diagnosis in the case of unclear tumors, e.g. if the primary tumor cannot be found, and the prognosis of the course of the disease. The object of tumor diagnosis is to increase the life span of the affected patient, improve his or her quality of life, but also to reduce the treatment costs and consequential costs.

Tumor markers are substances that are formed by the cancer itself or by the organism as a reaction to the growth of the tumor. Tumor markers occur in increased concentrations in the blood or in other bodily fluids, e.g. ascites, liquor or urine. Determining the concentration of tumor markers allows conclusions to be drawn about the presence, the course and the prognosis of tumor diseases. The measurable concentration of tumor markers in bodily fluids depends on, inter alia, the overall number of tumor cells (tumor mass), the synthesis rate of the tumor marker, the blood supply to the tumor and the marker-specific half-life. However, it is problematic that almost all currently identified tumor markers also occur in low but varying concentrations in healthy persons, and that it is not only malignant disease that can lead to an increase in the tumor markers. Furthermore, each tumor marker has substance-specific limitations.

The primary diagnosis of a tumor disease is generally diagnosed by a clinical examination, by imaging and/or endoscopic methods and/or a biopsy with subsequent pathologic clarification. Subsequently, the tumor-specific laboratory parameters are determined in vitro (in vitro diagnosis [IVD]) because the tumor aftercare already has to be planned at the time of the primary diagnosis. However, endoscopic methods and biopsies are invasive methods and therefore put stress on the patient, and furthermore are connected to high costs. Therefore, they are generally only considered in the case of a justified suspicion of a cancer disease or for the final assurance of the diagnosis. By contrast, in vitro diagnostic methods can be performed using various bodily fluids (e.g. urine or blood). Putting aside the minimal invasion connected with taking a blood sample, they are not or minimally invasive and more cost-effective than imaging and endoscopic in vivo methods.

The term “imaging methods” is understood to mean examination methods by which structures and organs in the body can be made visible. Examples of these include an examination using X-rays, computed tomography, magnetic resonance imaging, ultrasound diagnostics, scintigraphy, and positron emission tomography. Endoscopy also falls under imaging methods but, unlike the methods mentioned above, a probe has to be inserted into the body of the patient in this case. The disadvantage of imaging methods is that they are usually more expensive than in vitro diagnostic methods, and are often accompanied by a radiation load and therefore not suitable for screening a large asymptomatic collective.

A biopsy is an intervention into the human body—often under general anesthetics—in which a tissue sample is taken and subsequently evaluated in the laboratory. Often, particularly in the case of oncological diseases, it is only this examination that supplies the definite diagnosis. Since the biopsy is an invasive method often connected to pain and risks for the patient, the use thereof has to be meticulously deliberated. The biopsy is completely unsuitable for a screening of a broad asymptomatic collective.

The term “in vitro diagnostic agent” describes a medical product that is used as a reagent, reagent product, calibration material, control material, kit, instrument or system—individually or interconnected—for the in vitro examination of samples originating from the human or animal body, including donated blood and tissue. It is used to supply information relating to physiological or pathological conditions or to hereditary anomalies. Furthermore, it is used for checking harmlessness and compatibility in potential receivers of blood or tissue donations, or for monitoring therapeutic measures.

The article N Engl J Med 349:4 (2003), 335-342, the entire contents of which are hereby incorporated herein by reference, describes a screening test for prostate cancer using the prostate-specific antigen.

Thus, effective methods for the reliable diagnosis of tumor diseases are required, which, where possible, do not require invasive techniques. In particular, there is a need for a diagnosis, which is as early as possible, of such a disease in asymptomatic collectives and in groups at risk. Ideally, the diagnosis method should allow a prognostic statement and/or an assessment of the course and therapy.

### SUMMARY

In at least one embodiment, the present invention provides a diagnostic method that can diagnose a tumor disease as early as possible, i.e. preferably before the occurrence of clinically manifest symptoms and definitely before the occurrence of metastases. The method should be as sensitive as possible and therefore also be suitable for screening asymptomatic collectives without harboring the risk of overlooking a too large proportion of cancer patients. Furthermore, the method should be cost-effective and easy to carry out and be suitable for routine examinations to be carried out at regular intervals. Moreover, the method should also be suitable for monitoring the progress of the treatment of a cancer disease and for monitoring the therapeutic efficiency.

The method should be suitable for screening groups at risk, i.e. patients with an increased risk of a tumor disease due to their age, sex, history or family history; it should likewise be able to be used for completely unstratified patient collectives, that is to say for individuals without a known or suspected risk potential. Furthermore, the method should be simple to use, cost-effective and not put stress on the patient.

At least one embodiment of the invention is achieved by an in vitro method for diagnosing a tumor disease in a patient, comprising the following stages: (i) determining an IVD marker or an IVD marker panel in at least one biological sample of a patient, wherein the IVD marker has a high sensitivity to the tumor disease, (ii) determining the proportion of patients tested positive due to an adapted reference range of the IVD marker/IVD marker panel, wherein the reference range was adapted such that the number of individuals with false negative tests, the number of individuals with false positive tests and the number of individuals ultimately needing to be subjected to imaging diagnostics to clarify false negative and false positive results are balanced in respect of one another such that tumor screening can be carried out, possibly: (iii) deciding to carry out an imaging method specific to the respective tumor disease for clarifying possible false negative and/or false positive IVD results, or (iv) repeating stages (i) and (ii) after a defined time interval, or (v) carrying out an imaging method for imaging the tumor.

The term diagnostic “sensitivity” is understood to mean the proportion of true positive test results from the total number of all sick patients in percent. The sensitivity of tumor markers is on average at approximately 30% in the early stage of the disease and at approximately 70-90% in an advanced stage of the disease.

The term diagnostic “specificity” describes the proportion of true negative test results from the total number of all healthy people in percent.

The IVD markers used until now in tumor diagnostics, almost without exception, are distinguished by insufficient sensitivity and specificity. Although adjusting the reference range allows increasing the sensitivity to the detriment of the specificity or increasing the specificity to the detriment of the sensitivity, this does not allow a simultaneous increase in both sensitivity and specificity, and therefore a sufficient diagnostic performance for tumor screening to be obtained.

Surprisingly, it was found that tumors could be detected with high sensitivity by screening using IVD markers. While it was determined that a change in the reference range leads to an inversely related change in sensitivity and specificity, it was surprisingly determined that the parameters “sensitivity” and “specificity”, linked via the receiver operating characteristic, could be modeled such that a sufficient sensitivity to identify actual tumor patients, a sufficient reliability to exclude non-tumor patients, together with the costs for carrying out the IVD test or the IVD marker panel and the costs for subsequent imaging diagnostics for identifying the IVD false positives allow, in combination, an effective and, in terms of health economics, efficient screening method for finding tumor diseases. The result of such a test with an IVD marker or IVD marker panel allows a decision to be made as to whether an imaging method, an endoscopic examination, a targeted biopsy or a therapeutic intervention (e.g. a tumor ablation) should be carried out. The method according to an embodiment of the invention reduces the number of examinations to be carried out by the imaging method without changing the number of detectable tumors. This reduces both the socio-economic costs in the health sector and unnecessary exposure of patients to a radiation load.

### BRIEF DESCRIPTION OF THE DRAWINGS

Embodiments of the invention will be explained in more detail on the basis of the attached figures.

FIG. 1 shows the receiver operating characteristic (ROC) curve.

FIG. 2 shows a flowchart of the principle on which the in vitro method for diagnosing a tumor disease according to an embodiment of the invention is based.

FIG. 3 shows a flowchart for the screening or diagnostic method according to an embodiment of the invention on the basis of an asymptomatic patient collective.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

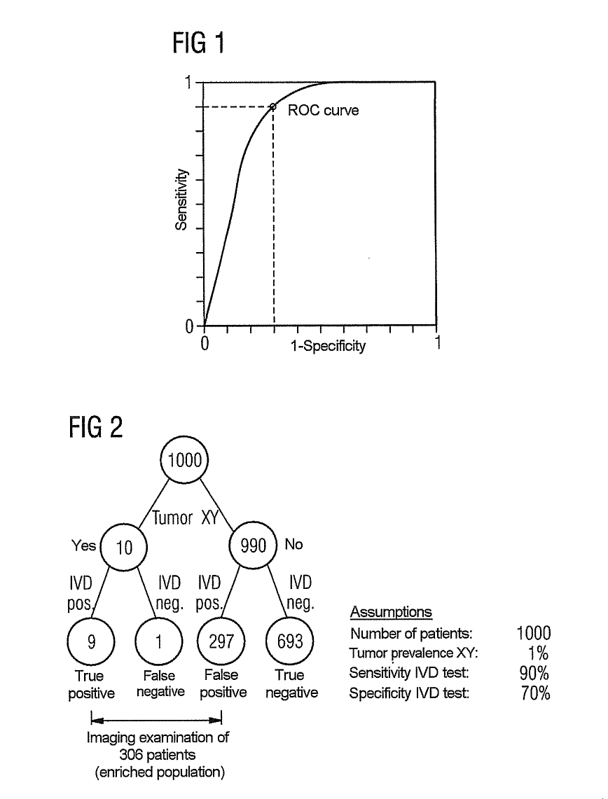

FIG. 1 shows the receiver operating characteristic (ROC) curve. This curve constitutes a method for evaluating analysis results. The relative frequency distribution resulting for each possible boundary value is determined, and the respectively associated sensitivity and specificity can be calculated therefrom.

FIG. 2 shows a flowchart of the principle on which the in vitro method for diagnosing a tumor disease according to an embodiment of the invention is based. A population of 1000 patients with a prevalence of tumor XY of 1% is examined, and the sensitivity of the in vitro test is assumed 90%. Examining the entire population in respect of a particular IVD marker leads to both positive and negative results in both groups. Thus, there are true positive and false negative results in the group of tumor carriers. The group of tumor-free patients contains both false positive and true negative results. The patients with a positive test result are now subjected to a continuative, i.e. imaging, examination in order ultimately to diagnose the possible presence of a tumor. The method according to an embodiment of the invention enriches true positive patients and so the number of patients who are unnecessarily subjected to an imaging examination can be minimized.

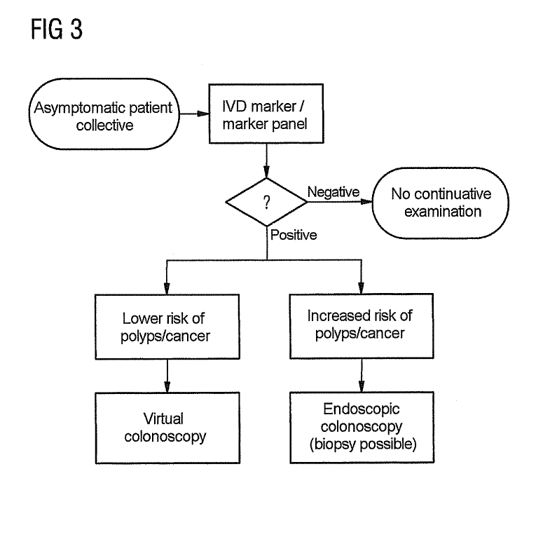

FIG. 3 shows how to carry out the method according to an embodiment of the invention, using the example of a colorectal cancer. FIG. 3 shows a flowchart for the screening or diagnostic method according to an embodiment of the invention on the basis of an asymptomatic patient collective. Only patients with a positive result in the in vitro test and an increased risk of polyps/cancer have to be subjected to an invasive examination, i.e. an endoscopic colonoscopy or biopsy.

Ideally, a tumor marker has both a high sensitivity and a high specificity. However, these two properties mutually exclude one another to a certain extent, as shown in the ROC (receiver operating characteristic) curve as per FIG. 1. The sensitivity and specificity of a marker can be set by changing the threshold (or cut-off) of the marker. This means that every IVD test can be set to have a high sensitivity to the detriment of the specificity.

In diagnostics, the ROC curve is a property of a marker. It is calculated from the analytical results of the measurement of a marker in a cohort by successively displacing the cut-off from the highest value in the cohort to the lowest value. Hence, the sensitivity-specificity pairings correspond to the patients in the cohort. If the cohort is sufficiently large, the steps in the ROC curve become invisible and a continuous, step-free (curved) line is obtained. Conversely, the specificity of the marker can be read from the curve for every possible sensitivity value—or conversely the sensitivity can be read for every possible specificity value.

The health-economic value of a diagnostic test is determined from the sensitivity and specificity thereof, the inherent costs thereof, the subsequent costs for clarifying the screening result (i.e., for example the costs for an MRI, a PET or a CT) and the QLYS (quality life years saved). Different pairings of sensitivity and specificity therefore result in a different health-economic value of the test. A low cut-off means that many (all) patients with a tumor are also tested as positive. The disadvantage thereof is that the specificity becomes correspondingly low and many false positive patients are subjected to a PET-CT. The result is that the clarification costs increase dramatically and the test becomes uneconomical. A high cut-off means that few tumors are tested positive, except for those already in advanced stages.

Hardly any patient is tested false positive; a PET (or the like) thus shows that (almost) every patient actually carries a tumor inside himself or herself. However, in actual fact only very few patients are identified at all in the screening and most tumors are overlooked. Hence, this test is of limited value: although few costs are incurred in the post-examination, only a small proportion of all patients are in fact identified and the money for the IVD screening test ultimately is mainly spent without being useful. A good pairing of sensitivity and specificity thus lies between these scenarios and can be placed into the most economical region with the aid of an ROC.

According to an embodiment of the invention, the reference range of a specific tumor marker now is adapted such that the number of individuals with false negative tests, the number of individuals with false positive tests and the number of individuals ultimately needing to be subjected to imaging diagnostics to clarify false negative and false positive results are balanced in respect of one another such that tumor screening can be carried out. To this end, the reference range of an IVD marker or the reference ranges of a plurality of IVD markers (marker panels) must be adapted together with an appropriate calculation specification such that the tumor disease can be detected with at least 80% certainty, preferably with at least 90% certainty and particularly preferably with at least 99% certainty.

According to an embodiment of the invention, those patients for whom an imaging method or a continuative examination and/or a therapy have been indicated are now enriched amongst the symptom-free patients. This means that an enrichment of true positive patients is obtained. This is obtained by using a very sensitive IVD test. The principle of the method according to an embodiment of the invention is illustrated in the attached FIG. 2.

According to an embodiment of the invention, a patient collective is screened using an IVD marker or an IVD marker panel with a high sensitivity and a suitable specificity, wherein patients with negative findings are excluded. As a result of the high sensitivity of the marker/the marker panel, only a few individuals with a tumor disease present are not identified. As a result of the adjusted specificity of the marker/the marker panel, the identified population also comprises false positives. Thus, the population fallen ill to the tumor as true positives is enriched compared to the general population. Due to the results obtained by this test, an informed decision now can be made as to whether continuative diagnostics, e.g. an imaging method or a tumor therapy, should be carried out. By adjusting the reference range of the IVD marker (the IVD markers), the sensitivity and specificity can be adapted in terms of an optimal health-economic value of the screening method.

Such a method has the advantage of being able to save costs in the health sector since IVD tests generally can be carried out more cost-effectively than imaging methods. Furthermore, systems for carrying out IVD tests are more widespread than devices for imaging methods. It is also easy to transport patient samples for carrying out a test to a distant laboratory, while it is more expensive and more complicated to request patients attend centralized screening centers for imaging diagnostics. This leads to a better patient cover or patient compliance. Furthermore, the radiation dose for the patient collective is reduced and patients are spared invasive methods such as an endoscopic colonoscopy or even a biopsy.

Using the method according to an embodiment of the invention, both benign and malignant tumor diseases, i.e. cancer diseases, can be diagnosed. The method is suitable for diagnosing all tumor diseases for which at least one tumor marker (IVD marker) is known. Examples of such known IVD markers, which, according to an embodiment of the invention, are preferably used, are specified in the following table. Moreover, every marker correlating with a tumor or cancer disease can be used. Such markers are known in the art.

| Tumor | Tumor marker |
|---|---|
| Colorectal polyps/adenoma | Metalloproteinase |
| Bladder cancer | NMP22 |
| Large bowel cancer | Carcinoembryonic antigen (CEA) |
| Prostate cancer | Prostate-specific antigen (PSA), |
| | cPSA, free PSA, TPS, sarcosine |
| Liver cancer | Alpha-fetoprotein (AFP) |
| Germinoma (testis, ovary) | chorionic gonadotropin (hCG) |
| | Alpha-fetoprotein (AFP) |
| Medullary thyroid carcinoma | Carcinoembryonic antigen |
| | (CEA) |
| | human calcitonin (hCT) |
| Papillary thyroid carcinoma | Thyroglobulin |
| Breast cancer | CA 15-3, CEA, CA 72-4 |

In vitro diagnostic test kits for determining tumor markers can be obtained for a number of diagnostic analysis systems and are carried out according to the specifications from the producer. The examination material—usually whole blood, serum, plasma, ascites, liquor, urine, feces, saliva, spinal fluid, nasal discharge, sputum, BAL, semen, breast discharge, wound discharge, gastric juices, sweat, or breath condensate—is processed in a known fashion. It is also possible to use combinations of tumor markers, so-called IVD marker panels. IVD marker panels are combinations of various laboratory parameters that correlate with the generation, the presence, the growth or the metastasis of a tumor. These various laboratory parameters are preferably measured in a biological sample of an asymptomatic patient, possibly in different samples of same individual as well (e.g. one parameter in blood plasma, a second parameter in urine). A marker panel distinguishes itself in that various properties of the primary disease are sampled, e.g. angiogenesis and apoptosis, wherein the combination of markers of the panel allows better diagnosis than would be possible using each of these individual markers separately.

The method according to an embodiment of the invention allows a reliable primary or differential diagnosis of a tumor disease in asymptomatic collectives or in groups at risk. As a result of the method according to an embodiment of the invention, therapy can be initiated and hence the tumor disease may possibly be cured. Depending on the quantity of the measured laboratory parameter, the method according to an embodiment of the invention may possibly also allow a statement to be made in relation to the severity or a prognosis to be made in relation to the progress of the tumor disease.

If the determination of the tumor marker or the determination of the tumor marker combined with an imaging method are reapplied after the tumor therapy has been initiated, the progress of the therapy after surgery or during radiotherapy, chemotherapy or hormone therapy can be assessed since a fall in the tumor marker levels indicates a reduction in the tumor mass. Moreover, the method can contribute to identifying those tumor patients who suffer from a relapse or tumor metastasis, in which the tumor marker level persists or even increases after therapy.

The method according to an embodiment of the invention is preferably suitable for diagnosing colorectal polyps/adenomas, large bowel cancer and prostate cancer.

FIG. 3 shows a flowchart for a potential screening method for detecting a colorectal cancer. An IVD marker or an IVD marker panel selects the patients for a subsequent imaging examination, as a result of which the number of imaging methods to be carried out is reduced. Patients with a positive IVD test result are further examined diagnostically, wherein an imaging method (either virtual or endoscopic colonoscopy) is used.

After identifying a patient with a positive test by determining an IVD marker or IVD marker panel, a decision can now be made as to whether an image-supported diagnosis method is carried out. In the process, present tumor tissue is imaged. In a separate step, a medical practitioner can then undertake a diagnostic evaluation. Examples of such methods, which may follow, are computed tomography, positron emission tomography, magnetic resonance imaging, ultrasound, etc. After carrying out such a method or instead of such a method, it is also possible to undertake a targeted biopsy, which provides information about the presence or lack of cancer disease after pathological clarification. Depending on the value obtained during the IVD test, a decision also can be made to carry out further IVD tests after a defined interval of time.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An in vitro method for diagnosing a tumor disease in a patient, comprising:
i) determining an IVD marker or an IVD marker panel in at least one biological sample of a patient, wherein the IVD marker has a relatively high sensitivity to the tumor disease;
ii) determining a proportion of patients tested positive due to an adapted reference range of the IVD marker or IVD marker panel, wherein the reference range is one adapted such that a number of individuals with false negative tests, a number of individuals with false positive tests and a number of individuals ultimately needing to be subjected to imaging diagnostics to clarify false negative and false positive results are balanced in respect of one another such that tumor screening can be potentially carried out; and one of
iii) deciding to carry out an imaging method specific to the respective tumor disease for clarifying at least one of possible false negative and false positive IVD results; or
iv) repeating (i) and (ii) after a defined time interval, or
v) carrying out an imaging method for imaging the tumor.

2. The method as claimed in claim 1, wherein the reference range of the IVD marker or IVD marker panel is one undertaken such that the tumor disease is detectable with at least 80% certainty, preferably with at least 90% certainty and particularly preferably with at least 99% certainty.

3. The method as claimed in claim 1, wherein the biological sample is a blood sample, a serum sample, a plasma sample, a urine sample, a fecal sample, a saliva sample, a spinal fluid sample, a nasal discharge sample, a sputum sample, a bronchoalveolar lavage sample, a semen sample, a breast discharge sample, a wound discharge sample, an ascites sample, a gastric juice sample or a sweat sample.

4. The method as claimed in claim 1, wherein the tumor disease is a colorectal polyp or an adenoma and the IVD marker is a metalloproteinase.

5. The method as claimed in claim 1, wherein the tumor disease is a thyroid carcinoma and the IVD marker is a carcinoembryonic antigen (CEA), human calcitonin (hCT) or thyroglobulin.

6. The method as claimed in claim 2, wherein the reference range of the IVD marker or IVD marker panel is one undertaken such that the tumor disease is detectable with at least 90% certainty.

7. The method as claimed in claim 6, wherein the reference range of the IVD marker or IVD marker panel is one undertaken such that the tumor disease is detectable with at least 99% certainty.

8. The method as claimed in claim 2, wherein the biological sample is a blood sample, a serum sample, a plasma sample, a urine sample, a fecal sample, a saliva sample, a spinal fluid sample, a nasal discharge sample, a sputum sample, a bronchoalveolar lavage sample, a semen sample, a breast discharge sample, a wound discharge sample, an ascites sample, a gastric juice sample or a sweat sample.

9. The method as claimed in claim 2, wherein the tumor disease is a colorectal polyp or an adenoma and the IVD marker is a metalloproteinase.

10. The method as claimed in claim 2, wherein the tumor disease is a thyroid carcinoma and the IVD marker is a carcinoembryonic antigen (CEA), human calcitonin (hCT) or thyroglobulin.

11. The method as claimed in claim 3, wherein the tumor disease is a colorectal polyp or an adenoma and the IVD marker is a metalloproteinase.

12. The method as claimed in claim 3, wherein the tumor disease is a thyroid carcinoma and the IVD marker is a carcinoembryonic antigen (CEA), human calcitonin (hCT) or thyroglobulin.

13. A computer readable medium including program segments for, when executed on a computer device, causing the computer device to implement the method of claim 1.

14. The method as claimed in claim 1, wherein the imaging method includes at least one of X-ray imaging, computed tomography imaging, magnetic resonance imaging, ultrasound imaging, scintigraphy, and positron emission tomography imaging.

15. The method as claimed in claim 1, wherein the imaging method includes endoscopy.

16. The method as claimed in claim 14, wherein the imaging method includes endoscopy.
